(19) United States
(12) Patent Application Publication
(10) Pub. No.: US 2003/0004485 A1
(43) Pub. Date: Jan. 2, 2003

(54) Breast pad

(76) Inventor: Shery D. Leeder, Toronto (CA)

Correspondence Address: RICHES, MCKENZIE & HERBERT, LLP, SUITE 1800, 2 BLOOR STREET EAST, TORONTO, ON, M4W 3J5, CA

(21) Appl. No.: 10/224,658

(22) Filed: Aug. 21, 2002

Related U.S. Application Data
(63) Continuation-in-part of application No. 09/487,349, filed on Jan. 19, 2000.

(30) Foreign Application Priority Data
Dec. 30, 1999 (GB) 9930824.9

(51) Int. Cl. A61F 13/15

(52) U.S. Cl. 604/385.07; 604/381

(57) ABSTRACT

A breast pad for absorbing fluid near a nipple is disclosed. The breast pad has an outer layer made from a substantially circular piece of absorbent material with a stitched radial tapering fold such as a dart which is deformable to a substantially concave or conical shape to substantially cover the nipple. The breast pad also has an intermediate layer of absorbing material having a shape which assists the intermediate layer to conform to an inside surface of the substantially concave shape of the outer layer. The intermediate layer is attached to the outer layer in a radial position corresponding to the stitched tapering fold such that the intermediate layer substantially covers the stitched tapering fold. In this way, the intermediate layer provides additional absorbent material near the nipple, and, prevents direct contact of the stitched tapering fold with the nipple. The breast pad also comprises an inner layer made of a hydrophilic material which dries substantially faster than the intermediate layer and draws fluid away from the nipple and towards the intermediate layer and the outer layer. The breast pad may have more than one outer layer to increase the fluid capacity of the breast. To assist in orienting the intermediate layer near the bottom of the nipple, the intermediate layer has a perceptively different color than the outer layer.

### RELATED APPLICATION

This application is a continuation-in-part of U.S. application Ser. No. 09/487,349 filed Jan. 19, 2000 and entitled “Breast Pad”.

### FIELD OF THE INVENTION

This invention relates to breast pads for absorbing fluid near the nipple of a breast. More particularly, this invention relates to breast pads which have a generally concave or conical shape to substantially cover a nipple.

### BACKGROUND OF THE INVENTION

Various types of breast pads have been used in the past to absorb fluid discharged from the nipple. In order to properly absorb the fluid near the nipple, it is generally necessary for the breast pad to have a substantially concave shape in order to fit comfortably and seamlessly around the nipple. Otherwise, if the breast pad does not have a concave shape, when the breast pad is placed over the nipple, the breast pad will create folds. The folds may irritate the nipple and may be visible through the bra and clothing of the wearer.

The prior art breast pads having a concave shape have been made in substantially two manners. The first manner is by heat treating the breast pad to create the appropriate concave shape or design. However, only certain types of material can be heat-treated so as to maintain the shape after the treatment. Furthermore, the types of materials which can be heat-treated tend not to be as absorbent as other types of material which cannot be heat-treated. In particular, terrycloth and other substantially cotton-based materials, which are very absorbent, cannot be heat-treated well.

The second manner for creating a breast pad having a concave shape is by sewing a “dart” into a substantially round material. The word “dart” used in this context refers to a stitched tapering fold in a garment. When a dart is sewed in a substantially circular material from the radius to the circumference of the circular material, the breast pad will then obtain a concave or conical shape.

It is apparent that a dart can be used to create a breast pad having a concave or conical shape regardless of the material used for the breast pad, and even if the breast pad is made from absorbent material. However, sewing a dart in a circular material will result in a breast pad having a seam along the dart. This seam in the breast pad may irritate the sensitive skin around the nipple. Furthermore, the stitching on the seam may act as a channel through which fluid may flow. Accordingly, while conical breast pads can be made from absorbent material by use of a dart, these breast pads suffer from the disadvantage that the seam from the dart may rub against the nipple and irritate the nipple, and may be visible through the bra and clothing of the wearer.

A further disadvantage of the prior art breast pads is that they can retain moisture near the nipple for extended periods of time. In other words, while the breast pad may work well to absorb fluid near the nipple, the fact that the moist breast pad continues to be in contact with the nipple may irritate the skin around the nipple.

Accordingly, there is a need in the art for a breast pad which has a concave shape made from absorbent materials which cannot necessarily be heat-treated, and yet does not expose the nipple to a seam caused by a dart. Furthermore, there is a need in the art for a breast pad which can keep the nipple substantially dry while still drawing fluid away from the nipple and absorbing fluid discharged from the nipple.

### SUMMARY OF THE INVENTION

Accordingly, it is an object of this invention to at least partially overcome the disadvantages of the prior art. Also, it is an object of this invention to provide an improved type of breast pad made of an absorbent material with a dart, but preventing direct contact of the nipple with the dart. Also, it is an object of this invention to provide a breast pad which, while being absorbent, does not expose the nipple to moisture for an extended period of time.

Accordingly, in one of its aspects, the present invention resides in a breast pad for absorbing fluid near a nipple, said breast pad comprising: an outer layer having a fastened tapering fold causing the outer layer to be deformable to a concave shape which substantially covers the nipple; an intermediate layer of absorbent material having a shape which assists the intermediate layer to conform to an inside surface of the concave shape of the outer layer; wherein the intermediate layer is attached to the outer layer such that the intermediate layer substantially covers the fastened tapering fold.

In a further aspect, the present invention resides in a breast pad having an inner layer separating the intermediate layer from the nipple, and wherein the inner layer dries substantially faster than the outer layer. To accomplish this, the inner layer comprises a material which is hydrophilic and may be made from a tetra channel fibre.

In a still further aspect, the present invention resides in a breast pad for absorbing fluid near a nipple, said breast pad comprising an outer layer of absorbent material having a fastened tapering fold causing the outer layer to be deformable to a concave shape which substantially covers the nipple, said fastened tapering fold forming a seam on an inside surface of the outer layer; an intermediate layer of absorbent material having a shape which assists the intermediate layer to conform to an inside surface of the concave shape of the outer layer; an inner layer for contacting the nipple; wherein the intermediate layer is attached to the outer layer such that the intermediate layer substantially covers the fastened tapering fold to substantially prevent the nipple from rubbing against the seam of the tapering fold through the inner layer; and wherein the inner layer comprises a material which draws fluid from the nipple to be absorbed by the outer layer and intermediate layer.

Accordingly, one advantage of the present invention is that the breast pad has a concave shape and is made from an absorbent material, such as terrycloth, however the seam from the dart is not in direct contact with the nipple. In this way, the nipple is protected from the dart by the intermediate layer. Furthermore, the intermediate layer has a shape which conforms to the inner surface of the outer layer when the outer layer has a conical shape. In this way, not only does the intermediate layer protect the nipple against the seam of the dart, the intermediate layer will not itself create folds, which folds may irritate the nipple and may be visible through the bra and clothing of the wearer.

A further advantage of the present invention is that the intermediate layer and the outer layer can both absorb fluid. In other words, the breast pad will have an intermediate layer, which may be made of an absorbent material, as well as an outer layer which may also be made of an absorbent material. In one embodiment, additional outer layers, could be used to increase absorbency. Preferably, the intermediate layer will absorb most of the moisture so that no leakage occurs through the outer layer to the clothing. To further assist in having the intermediate layer absorb fluid, it is preferred that the intermediate layer be oriented such that it is at the lower end of the breast pad when the breast pad is next to the nipple. In this way, most fluid will flow by gravity to the intermediate layer. To facilitate orienting the intermediate layer, the intermediate layer is preferably of a different colour than the outer layer so that it can be easily oriented at the bottom of the nipple.

A further advantage of the present invention is that the breast pad may comprise an inner layer made from a hydrophilic material which draws fluid away from the nipple and towards the intermediate layer. In this way, the inner layer performs the dual function of drawing fluid away from the nipple, thereby keeping the nipple dry, and, drawing fluid towards the intermediate layer, thereby preventing leakage from the breast pad. In the embodiment where more than one outer layer is used, the additional outer layer or layers can absorb excess fluid thereby assisting the hydrophilic material to draw fluid away from the nipple.

A further advantage is that, unlike other types of breast pads, the breast pad according to the present invention can be reusable. This is the case at least because the breast pad may be made from absorbent material, such as terrycloth, which can be washed and reused. In this way, the breast pad according to the present invention is substantially economical as it can be reused, and, it is environmentally friendly as compared to breast pads which are disposable.

Further aspects of the invention will become apparent upon reading the following detailed description and drawings which illustrate the invention and preferred embodiments of the invention.

### BRIEF DESCRIPTION OF THE DRAWINGS

In the drawings, which illustrate embodiments of the invention:

FIG. 1 shows a top view of the outer layer of the breast pad according to one embodiment of the invention;

FIG. 2 shows a top view of the intermediate layer of the breast pad according to one embodiment of the invention;

FIG. 3 shows a top view of the inner layer of the breast pad according to one embodiment of the invention;

FIG. 4 shows a top view of an assembled breast pad according to one embodiment of the present invention showing the inner layer, the intermediate layer and the outer layer;

FIG. 5 shows a top view of an additional outer layer of the breast pad according to a further embodiment of the invention; and

FIG. 6 shows a top view of an assembled breast pad according to a further embodiment of the present invention showing the inner layer, the intermediate layer, the outer layer and at least one additional outer layer.

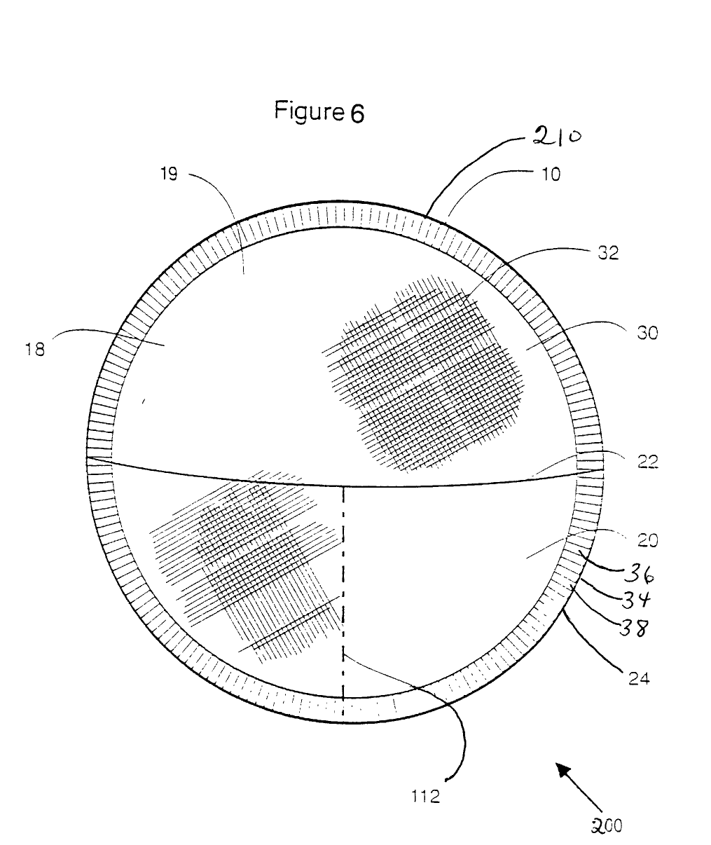

### DETAILED DESCRIPTION OF THE PREFERRED EMBODIMENTS

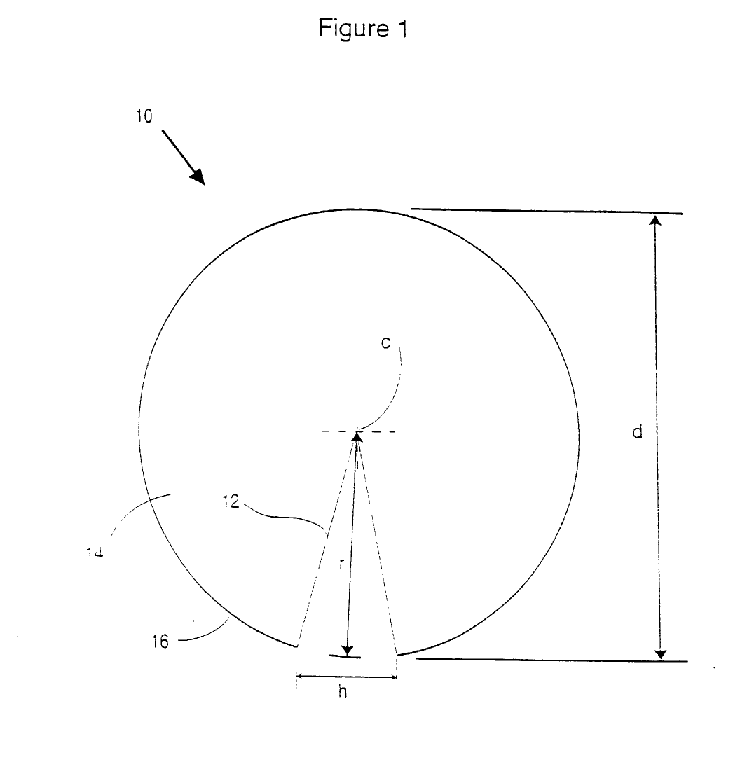

FIG. 1 shows the outer layer 10 according to one embodiment of the present invention. As shown in FIG. 1, the outer layer 10 is formed of a material which is preferably absorbent and initially has a substantially circular shape. Preferably, the diameter d of the outer layer 10 will be between four and six inches (100 and 150 millimeters), and more preferably five inches (125 millimeters). The radius r will therefore be between two and three inches (50 and 75 millimeters) and more preferably two and a half inches (37.5 millimeters).

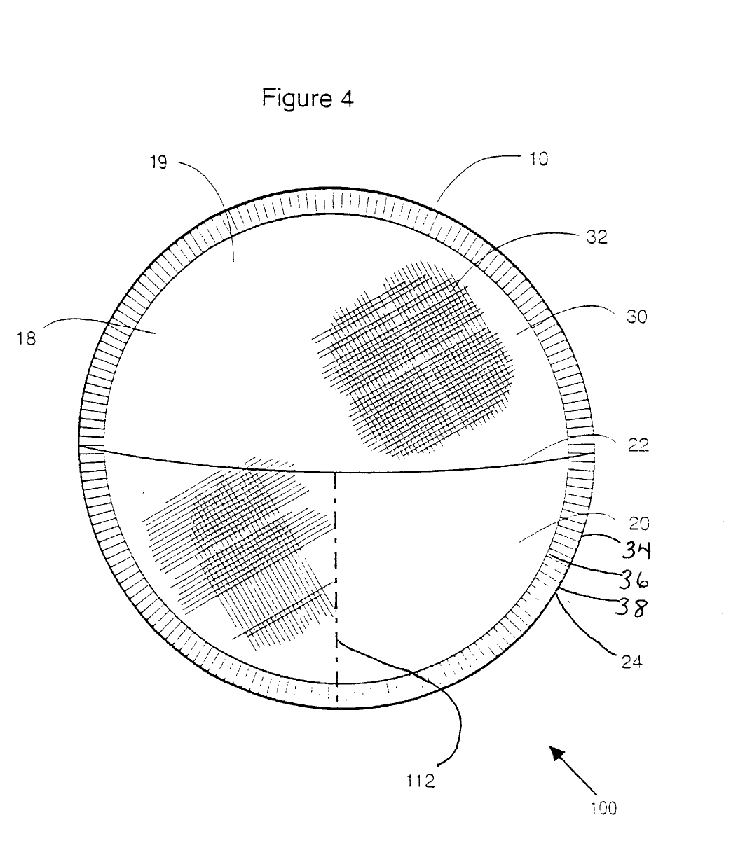

As also shown in FIG. 1, a dart 12 is made in the outer layer 10. The dart 12 is created by removing a sector of the outer layer 10 and then fastening, such as by stitching the sector to form a stitched tapering fold (shown in FIG. 4 by reference numeral 112). In the embodiment where the outer layer 10 is circular, the stitched tapering fold will extend along the radius r. Preferably, the sector will extend along the circumference for a distance h of preferably one half inch (12.5 millimeters) to one and a half inches (37.5 millimeters) and more preferably one inch (25 millimeters).

Once the dart 12 is stitched along the tapering fold, the outer layer 10 will have a fastened tapering fold which will be deformable to a substantially concave shape. In particular, in the embodiment as shown in FIG. 1 where the outer layer 10 is formed from a substantially circular shape, the outer layer 10 will have a marginally conical shape when the dart 12 is sewed. In this way, the outer layer 10 will be deformable to a concave or conical shape which will substantially cover the nipple of the breast.

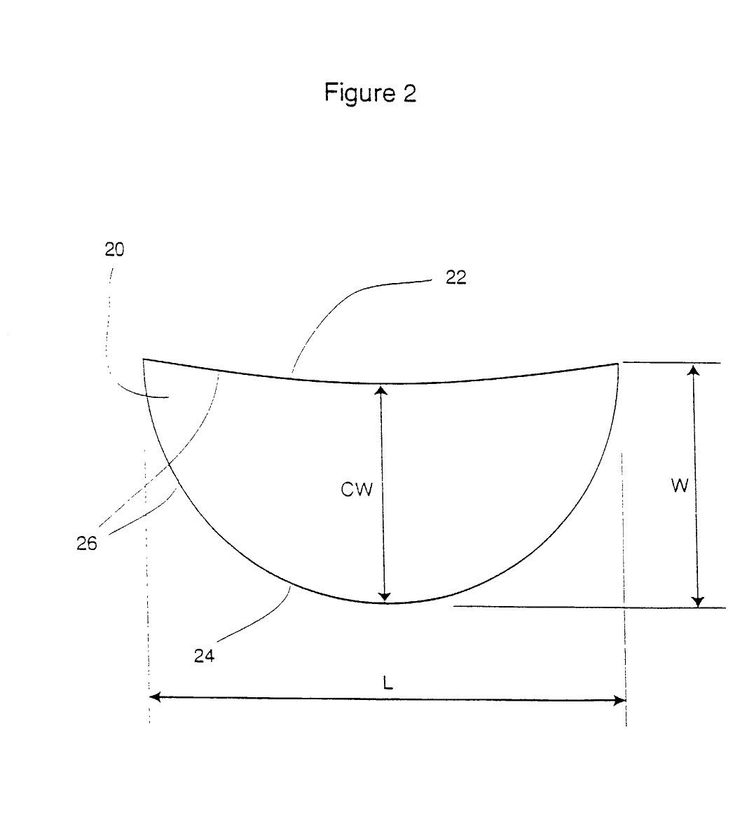

FIG. 2 shows an intermediate layer 20 of absorbent material according to an embodiment of the present invention. As shown in FIG. 2, the intermediate layer 20 will have a length 1 which preferably is substantially equivalent to the diameter d of the outer layer 10. More preferably, the intermediate layer 20 will have a width w which is approximately equivalent to the radius r of the outer layer 10. However, the intermediate layer 20 will preferably have centre width cw which is less than the outside width w. This is done in part so that the intermediate layer 20 will have a shape 26 which assists the intermediate layer 20 to conform to an inside surface 14 of the substantially concave shape of the outer layer 10.

As also shown in FIG. 2, the intermediate layer 20 is preferably formed from a concave edge 22 intersecting a convex edge 24. This is preferable in the embodiment where the outer layer 10 is formed from a circular material so that the intermediate layer 20 will conform to the inside surface 14 of the conical shape formed by the outer layer 10. In particular, it is preferred that the convex edge 24 closely follow the circumference 16 of the outer layer 10 when the dart 12 has been fastened, such as by sewing. In other words, when the concave shape of the outer layer 10 is substantially conical, it is preferred that the intermediate layer 20 have a substantially crescent shape 26 to conform to the inside surface 14 of the conical shape and so that the intermediate layer 20 can be attached along the circumference 16 of the outer layer 10.

Once the tapering fold 112 is fastened, such as by stitching, the outer layer 10 will be deformable to a substantially concave shape which covers the nipple. At this time, the intermediate layer 20 can be attached to the outer layer 10. The intermediate layer 20 is attached to the outer layer 10 such that the intermediate layer 20 will substantially cover the tapering fold 112. In other words, the centre width cw should be sufficient to substantially cover or occlude the seam of the fastened or stitched tapering fold caused by sewing or otherwise fastening the dart 12 of the outer layer 10 together. In this way, the intermediate layer 20 will provide absorbent material intermediate the nipple and the outer layer 10 at the location of the stitched tapering fold 112. This will prevent the seam of the stitched tapering fold 112 from contacting the nipple or, rubbing against the nipple, through an inner layer 30 discussed below. Therefore, the intermediate layer 20 will prevent fluid from flowing within channels formed by the seam of the stitched tapering fold 112 and will prevent the stitched tapering fold 112 from irritating the nipple by rubbing against the nipple.

To ensure that the intermediate layer 20 does not itself fold within the inside surface 14 of the concave shape of the outer layer 10, the intermediate layer 20 preferably has a shape 26 which assists the intermediate layer 20 to conform to the inside surface 14 of the concave shape of the outer layer 10. In this way, the intermediate layer 20 forms a seamless seal of absorbent material against the nipple without any folds to irritate the nipple or act as a channel for fluid. Moreover, the intermediate layer 20 protects the nipple from the stitched tapering fold.

It is understood that the intermediate layer 20 can have any shape which assists the intermediate layer 20 to conform to an inside surface 14 of the substantially concave shape of the outer layer 10. While FIG. 2 shows particular dimensions and a crescent shape 26, it is understood that the invention is not restricted to an intermediate layer 20 having this particular shape 26. Rather, the intermediate layer 20 can have any shape 26 which can substantially cover the stitched tapering fold (shown in FIG. 4 by reference numeral 112) while conforming to the inside surface 14 of the concave outer layer 10. It is also understood that the shape 26 of the intermediate layer 20 shown in FIG. 2 is the preferred shape for an outer layer 10 formed from a circular material and has a concave shape which is marginally conical. However, the intermediate layer 20 may have other shapes 26, including rectangles, triangles, sections of circles, such as sectors and sections of cones. It is also understood that some of these other shapes may be more preferable in different situations, depending on the specific concave shape of the outer layer 10.

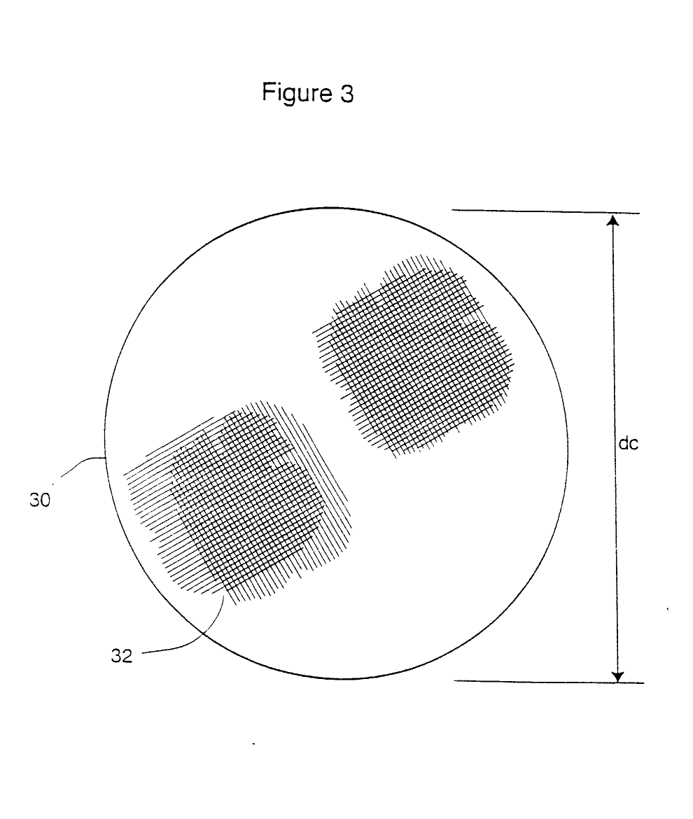

FIG. 3 shows an inner layer 30 according to one embodiment of the present invention. Preferably, the inner layer 30 has a diameter dc which closely corresponds to the diameter d of the outer layer 10 after the stitched tapering fold has been made. The inner layer 30 is designed to be in contact with the nipple and separate the intermediate layer 20 and the outer layer 10 from the nipple.

The inner layer 30 preferably is formed from a material which helps to draw fluid away from the nipple and towards the intermediate layer 20 and the outer layer 10. In a preferred embodiment, the inner layer 30 is formed of a hydrophilic material to facilitate drawing fluid away from the nipple and towards the intermediate layer 20. Such hydrophilic materials can include materials which have tetra channel fibres that draw fluid away from the nipple. The channels speed moisture along the fibres to the outer surface and the perimeter of the inner layer 30. Preferred examples of hydrophilic materials that have tetra channel fibres include DUPONT COOLMAX (trade mark) performance fabrics. The hydrophilic inner layer 30 draws the fluid away from the nipple to be absorbed, and therefore stored, in the intermediate layer 20 and outer layer 10. The fluid or moisture can be stored in the intermediate layer 20 and outer layer 30 until the pad is removed.

Preferably, to increase the breathability of the inner layer 30, the inner layer 30 will have a mesh format 32. Preferably, the openings will be in the range of one half to two millimeters in diameter. More preferably, the mesh format 32 will have openings of about one millimeter in diameter.

Because of the hydrophilic features of the inner layer 30, the inner layer 30 dries substantially faster than the intermediate layer 20 or the outer layer 10. In this way, the hydrophilic inner layer 30 maintains the nipple substantially dry and separates it from the moist intermediate layer 20 and outer layer 10. The hydrophilic inner layer 30 is relatively thin such that it can conform to the shape of the nipple substantially seamlessly. If any folds are created by the hydrophilic inner layer 30, because the inner layer 30 is relatively thin, these folds would be relatively small so as to substantially seamlessly contact the nipple thereby avoiding irritation. The hydrophilic inner layer 30 assists in drawing fluid away from the nipple and towards the absorbent intermediate layer 20 and one or more outer layers 10.

FIG. 4 shows the breast pad 100 according to one embodiment of the present invention. As shown in FIG. 4, the breast pad comprises the outer layer 10 with the stitched tapering fold 112 having been stitched, as shown generally by the dot-dash line marked with reference numeral 112. As also shown in FIG. 4, the intermediate layer 20 has been attached to the outer layer 10 such that the intermediate layer 20 substantially covers or occludes the stitched tapering fold 112. In this way, the intermediate layer 20 will protect the nipple from the seam of the stitched or fastened tapering fold 112.

As also shown in FIG. 4, the intermediate layer 20 will preferably have the convex edge 24 attached along the circumference 16 of the outer layer 10 to ensure that the intermediate layer 20 is kept in a position which substantially covers the stitched tapering fold 112. Furthermore, the convex edge 24 and the concave edge 22 of the intermediate layer 20 will assist the intermediate layer 20 to conform to the inside surface 18 of the outer layer 10. In this way, the intermediate layer 20 will not itself create folds.

Attached on top of the intermediate layer 20 is the inner layer 30. The inner layer 30 will preferably have meshes 32 as described above. The inner layer 30 will separate the intermediate layer 20 and also a part 19 of the outer layer 10 from the nipple. The conical shape of the outer layer 10 keeps the inner layer 30 substantially in contact with the nipple.

Preferably, the intermediate layer 20 is made from an absorbent material such as terrycloth. More preferably, the intermediate layer 20 is made from terrycloth having at least 94% cotton and preferably 100% cotton. Likewise, in order to further increase the absorbency of the breast pad, as well as the capacity for storing moisture, it is preferred that the outer layer 10 also be made from an absorbent material. Preferably, both the outer layer 10 and the intermediate layer 20 are made from a terrycloth material having at least 94%, and preferably 100%, cotton. More preferably, to ensure that the outer layer 10 has sufficient strength to deform to and maintain the concave shape and has sufficient durability, it is preferred that the outer layer 10 be made from a terrycloth material having at least 18 ounces per linear meter.

Accordingly, the breast pad 100 shown in FIG. 4 provides a hydrophilic inner layer 30 which is in contact with the nipple and draws moisture and fluid from the nipple towards the absorbent intermediate layer 20 and outer layer 10. The hydrophilic inner layer 30 is preferably made from a thin material, and may have meshes 32 such that it would not create large folds and substantially seamlessly contact the nipple. While the outer layer 10 may be made of thick terrycloth material, thereby forming a relatively large seam along the fastened tapering fold 112, the seam formed by the fastened tapering fold 112 will be substantially covered or occluded by the intermediate layer 20 such that the nipple would not become irritated by rubbing against the seam of the fastened tapering fold 112 through the relatively thin hydrophilic inner layer 30. In addition, the intermediate layer 20 is preferably formed from a shape such that it can conform against the inside surface 18 of the outer layer 10. The breast pad 100 may also have stitching 34 to attach the inner layer 30, intermediate layer 20 and outer layer 10 together. To the extent the stitching 34 has a seam 36, this would be located circumferentially, rather than radially, and at a distance from the centre of the nipple. Radial stitching 38 may extend for a short distance to ensure that all the components of the breast pad 100 have been properly and reliably stitched together.

In this way, the breast pad 100 provides a hydrophilic inner layer 30 in contact with the nipple 30 to maintain the nipple dry. In addition, the inner layer 30 is generally made of thin material so it can substantially seamlessly contact the nipple. The conical shape of the outer layer 10, and to some extent the inner layer 20, assist in maintaining the hydrophilic inner layer 30 in contact with the nipple. To the extent that the thicker outer layer 10 has a fastened tapering fold 112, the intermediate layer 20 protects the nipple from contacting or rubbing against the fastened tapering fold 110 to avoid irritation. Furthermore, the intermediate layer 20 avoids fluid running down the seam formed by the fastened tapering fold 112 and either pooling at the bottom of the breast pad or flowing out from the breast pad 100. Therefore, the combination of the hydrophilic inner material 30 to draw fluid away from the nipple thereby moving moisture away from the nipple to keep it relatively dry, along with the absorbent outer layer 10 to absorb fluid and keep the inner layer 30 in contact with the nipple, as well as the intermediate layer 20 to further absorb fluid and protect the nipple from rubbing against the fastened tapering fold keeps the nipple dry and substantially prevents irritation.

Accordingly, the intermediate layer 20 and outer layer 10 absorb and hold the fluid until the breast pad 100 can be replaced. In general, the breast will be placed adjacent an interior surface of a cup-like portion of a bra or a bra-like garment. The centre c of the outer layer 10 will generally be placed over the woman's nipple.

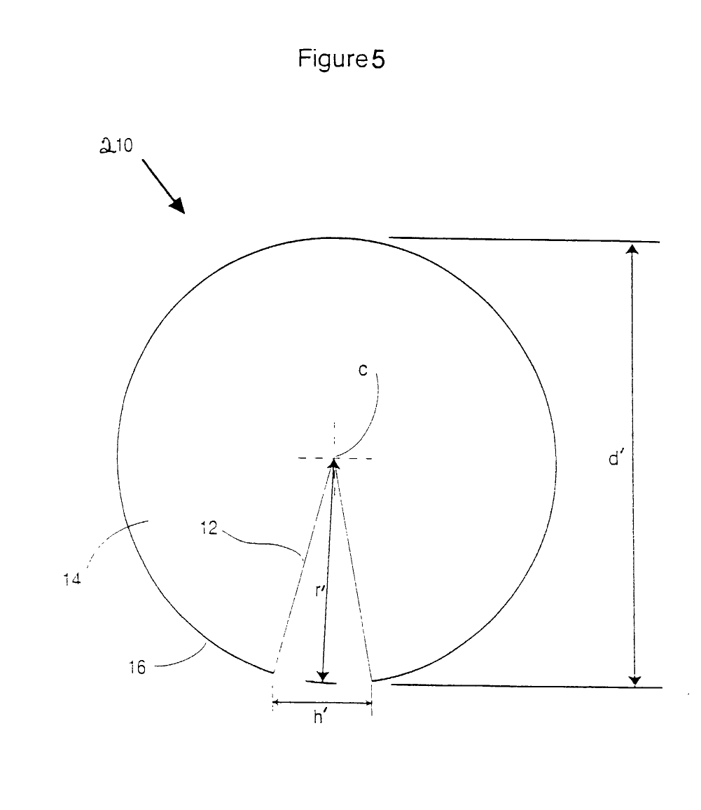

In order to increase absorbency and fluid capacity of the breast pad 100, additional layers may be used. These additional layers can be placed on the exterior surface of the outer layer 10 and be sewn onto the outer layer 10 by the stitching 34. These additional outer layers can be similar in construction to the outer layer 10, as shown in FIG. 1, or alternatively, they may have different construction and have different dimensions as shown by reference numeral 210 in FIG. 5, both of which are collectively referred to as external outer layers 10, 210. These external outer layers 10, 210 can be used to absorb and hold additional fluid. In the case where different dimensions are used, as shown by additional outer layer 210 in FIG. 5, generally the diameter d′ and r′ will be larger so that it can more easily fit over the outer layer 10. It is understood that more than one external outer layer 10, 210 could be added, and the dimensions may be of an ever increasing diameter d′ or, the same diameter d as shown in FIG. 1. In either case, the external outer layer will provide increased absorbency and fluid capacity. In each case, the intermediate layer 20, outer layer 10 and external outer layers 10, 210, are preferably attached along the circumference 24 by stitching 34 such that the hydrophilic inner layer 30 can draw fluid from the nipple to be absorbed by the intermediate layer 20, outer layer 10 and external outer layer 10, 210.

It is apparent that gravity will act on any fluid to cause it to flow downwards. Accordingly, it is preferred that the breast pad 100 be oriented against the nipple such that the intermediate layer 20 is located at the bottom portion of the nipple. In order to assist in orienting the breast pad 100 in this manner, it is preferred that the intermediate layers 20 have a colour which is perceptively different from the outer layer 10. In this way, the portion of the breast pad 100 where the intermediate layer 20 is attached will be immediately discernible. Furthermore, if the inner layer 30 has meshes 32, the colour of the intermediate layer 20 and the outer layer 10 will be immediately apparent through the meshes 32 of the inner layer 30.

It is understood that while the present invention has been described and illustrated above with respect to one preferred embodiment having an outer layer 10 formed from a substantially circular material and deformable to a marginally conical shape, any outer layer 10 having any initial shape that is deformable into a concave shape could be used. Furthermore, it is understood that the intermediate layer 20 is not restricted to the particular crescent shape illustrated in FIG. 2. Rather, the intermediate layer 20 can have any shape including triangular, rectangular, square, linear, or a section of a circle or cone, such as a sector, can be used so that the intermediate layer 20 conforms to the inside surface 14 of the concave shape of the outer layer 10.

It is understood that while the disclosure has been described in terms of a stitched tapering fold 112, the invention is not limited to a tapering fold which has been stitched. Rather, the invention includes all embodiments where the tapering fold has been stitched, glued, stapled or otherwise fastened together.

It will be understood that, although various features of the invention have been described with respect to one or another of the embodiments of the invention, the various features and embodiments of the invention may be combined or used in conjunction with other features and embodiments of the invention as described and illustrated herein.

Although this disclosure has described and illustrated certain preferred embodiments of the invention, it is to be understood that the invention is not restricted to these particular embodiments. Rather, the invention includes all embodiments which are functional, electrical or mechanical equivalents of the specific embodiments and features that have been described and illustrated herein.

The embodiments of the invention in which an exclusive property or privilege is claimed are defined as follows:

1. A breast pad for absorbing fluid near a nipple, said breast pad comprising:
an outer layer of absorbent material having a fastened tapering fold causing the outer layer to be deformable to a concave shape which substantially covers the nipple, said fastened tapering fold forming a seam on an inside surface of the outer layer;
an intermediate layer of absorbent material having a shape which assists the intermediate layer to conform to an inside surface of the concave shape of the outer layer;
an inner layer for contacting the nipple;
wherein the intermediate layer is attached to the outer layer such that the intermediate layer substantially covers the fastened tapering fold to substantially prevent the nipple from rubbing against the seam of the fastened tapering fold through the inner layer; and
wherein the inner layer comprises a material which draws fluid from the nipple to be absorbed by the outer layer and intermediate layer.

2. The breast pad as defined in claim 1 wherein the inner layer is formed of a hydrophilic material which draws fluid from the nipple towards the outer layer and intermediate layer; and
wherein the concave shape of the outer layer maintains the inner layer substantially in contact with the nipple to draw fluid from the nipple to the outer layer and intermediate layer.

3. The breast pad as defined in claim 2 further comprising:
at least one external outer layer made of an absorbent material and attached to the external surface of the outer layer, said external outer layer deformable to the concave shape of the outer layer to substantially cover the exterior surface of the outer layer.

4. The breast pad as defined in claim 3 wherein the hydrophilic inner layer is attached to the intermediate layer, outer layer and external outer layer to draw fluid away from the nipple and towards the absorbent intermediate layer, outer layer and external outer layer.

5. The breast pad as defined in claim 1 wherein the concave shape is substantially conical and wherein the shape of the intermediate layer has a convex edge intersecting a concave edge with the convex edge fastened to a circumference of the conical outer layer near the fastened tapering fold.

6. The breast pad as defined in claim 1 wherein the concave shape is substantially conical and wherein the shape of the intermediate layer is substantially crescent shaped, and conforms to the inside surface of the conical shape of the outer layer, such that the intermediate layer and the conical shape of the outer layer keep the inner layer in contact with the nipple.

7. The breast pad as defined in claim 6 wherein the intermediate layer has a first colour and the outer layer has a second colour perceptively different from the first colour.

8. The breast pad as defined in claim 1 wherein the inner layer dries substantially faster than the intermediate layer and outer layer.

9. The breast pad as defined in claim 6 wherein the inner layer comprises a material having tetra channel fibre to draw fluid away from the nipple and towards the intermediate layer and outer layer.

10. The breast pad as defined in claim 1 wherein the inner layer is made from substantially 100% polyester.

11. The breast pad as defined in claim 1 wherein the inner layer has a mesh format with openings of about 0.5 to 1.5 millimeters in diameter.

12. The breast pad as defined in claim 4 wherein the outer layer is formed from an absorbent material having a substantially circular shape with a centre and a circumference and wherein the fastened tapering fold extends from about the centre to the circumference of the material; and
wherein the intermediate layer, outer layer and at least one external outer layer are attached along the circumference of the outer layer such that the hydrophilic inner layer draws fluid from the nipple to be absorbed by the intermediate layer, outer layer and external outer layer.

13. The breast pad as defined in claim 12 wherein the outer layer at least one external layer and the intermediate layer are made of a terrycloth material.

14. The breast pad as defined in claim 13 wherein the terrycloth material consists of at least 94% cotton.

15. The breast pad as defined in claim 14 wherein the terrycloth material of the outer layer and external outer layer is at least 18 ounces per linear meter.

16. The breast pad as defined in claim 1 wherein the shape of the intermediate layer is selected from the group comprising rectangles, triangles, sections of a circle and sections of a cone.

17. The breast pad as defined in claim 1 wherein the tapering fold is fastened by stitching.

18. A breast pad for absorbing fluid near a nipple, said breast pad comprising:
an outer layer having a stitched tapering fold causing the outer layer to be deformable to a concave shape which substantially covers the nipple;
an intermediate layer of absorbent material sufficiently large to occlude the stitched tapering fold;
an inner layer of a hydrophilic material for contacting the nipple to draw fluid from the nipple to be absorbed by the intermediate layer.

19. The breast pad as defined in claim 18 wherein the outer layer comprises an absorbent material for storing fluid; and
wherein the conically shaped outer layer keeps the inner layer substantially seamlessly in contact with the surface of the nipple to draw fluid from the nipple for absorption by the intermediate layer and the outer layer.

20. The breast pad as defined in claim 19 further comprising:
at least one external outer layer made of an absorbent material and attached to the external surface of the outer layer, said external outer layer deformable to the concave shape to substantially cover the exterior surface of the outer layer.
